(19) United States
(12) Patent Application Publication
(10) Pub. No.: US 2025/0375307 A1
(43) Pub. Date: Dec. 11, 2025

(54) CORRECTIVE DEVICE FOR RELIEVING NECK AND SHOULDER PAIN

(71) Applicant: Esther Lopez, Cherry Hill, NJ (US)

(72) Inventor: Esther Lopez, Cherry Hill, NJ (US)

(21) Appl. No.: 19/215,217

(22) Filed: May 21, 2025

Related U.S. Application Data
(60) Provisional application No. 63/650,242, filed on May 21, 2024.

(51) Int. Cl. A61F 5/01; A47G 9/10

(52) U.S. Cl. CPC A61F 5/01; A47G 9/1063; A47G 9/1081

(57) ABSTRACT

A corrective device for relieving pain in the neck, shoulder, and upper back regions of the human body. More particularly, the corrective device comprises two arm support foam pads, a neck support pad, and a tummy support pad, each of the four support pads having adjustable straps to accommodate different body sizes and varying arm positions. In operation, the user lies face down between the arm support pads, positions their arms on the support pads with elbows on the pads, places their chin on the neck support pad, and positions the tummy support pad under their navel. The adjustable straps allow for customization to the user's body dimensions. The device may provide relief from neck, shoulder and upper back pain, and may correct conditions such as Kyphosis Hump and Rounded Shoulders.

### CROSS-REFERENCE TO RELATED APPLICATIONS

This application is a non-provisional patent application claiming the benefit of U.S. Provisional Patent Application No. 63/650,242, filed on May 21, 2024, entitled “CORRECTIVE DEVICE FOR RELIEVING NECK, SHOULDER, AND UPPER BACK PAIN,” the entire disclosure of which is hereby incorporated by reference herein.

### FIELD OF THE INVENTION

The present invention relates to a corrective device for relieving pain in the neck, shoulder, and upper back regions of the human body. The present disclosure relates to therapeutic devices for correcting posture and alleviating pain in the musculoskeletal system. More specifically, it relates to a supportive positioning device designed for relieving pain and correcting postural abnormalities in the cervical, thoracic, and scapular regions while a user lies in a prone position.

### BACKGROUND OF THE INVENTION

Devices are well known in the prior art for alleviating pain from the neck, shoulder, and upper back regions of the human body, ranging from neck pillows to restrictive strapping devices. Many conventional therapeutic devices focus on providing passive support to the affected areas rather than facilitating active correction of postural abnormalities. Neck pillows, for instance, are designed to support the cervical spine while sleeping in a supine position but do not address the underlying muscular imbalances that contribute to chronic pain. Similarly, restrictive strapping devices often immobilize the affected area, which may provide temporary relief but can lead to muscle atrophy and increased dependency on external support over time.

Kyphosis, commonly known as “Dowager's Hump” or “Widow's Hump,” is a condition characterized by excessive curvature of the thoracic spine, resulting in a rounded upper back appearance. This postural abnormality is prevalent among older adults, particularly women, and can cause significant pain and functional limitations. Traditional approaches to managing kyphosis include physical therapy, bracing, and pain medications, each with varying degrees of effectiveness and limitations.

Rounded shoulders, another common postural issue, occur when the shoulders roll forward from their neutral alignment. This condition is often associated with prolonged sitting, computer use, and other activities that promote a forward head posture. Over time, rounded shoulders can lead to muscle imbalances, with the anterior chest muscles becoming tight and shortened while the posterior shoulder and upper back muscles become weak and elongated.

Current treatment modalities for these postural abnormalities typically require extended periods of therapy before noticeable improvements occur. Physical therapy regimens often necessitate multiple weekly sessions over several months, with outcomes heavily dependent on patient compliance with prescribed exercises. Bracing approaches, while providing external support, may not effectively retrain the musculature needed for long-term postural correction.

The scapular region, comprising the shoulder blades and surrounding musculature, plays a crucial role in upper body mechanics and posture. Dysfunction in this area can contribute to a cascade of compensatory movements that exacerbate pain and postural abnormalities. Many existing therapeutic devices fail to adequately address scapular positioning and the integrated nature of the cervical-thoracic-scapular complex.

Prone positioning therapy, where the patient lies face down, has shown promise in addressing certain musculoskeletal issues by utilizing gravitational forces to assist in postural realignment. However, standard prone positioning without proper support can create discomfort in the neck, breathing difficulties, and pressure on sensitive abdominal structures, limiting its therapeutic potential and patient tolerance.

The limitations of existing approaches highlight the need for comprehensive therapeutic solutions that address the multifaceted nature of postural abnormalities and associated pain patterns. An effective approach would ideally provide appropriate support while facilitating active correction of muscular imbalances in a comfortable, sustainable manner that promotes patient compliance.

### SUMMARY OF THE INVENTION

The present subject matter relates to a corrective device for relieving pain in the neck, shoulder, and upper back regions of the human body. More particularly, the corrective device of the present subject matter comprises two arm support foam pads, a neck support pad, and a tummy support pad, each of the four support pads having adjustable straps to accommodate different body sizes and varying arm positions. The present subject matter relates to a corrective device for relieving pain in the neck, shoulder, and upper back regions of the human body. More particularly, the corrective device of the present subject matter comprises two arm support foam pads, a neck support pad, and a tummy support pad, each of the four support pads having adjustable straps to accommodate different body sizes and varying arm positions.

A corrective device may comprise a left arm support pad and a right arm support pad, each arm support pad having an upper end, a lower end, a first angled edge extending outwardly and downwardly from the upper end, a second angled edge extending inwardly and downwardly to the lower end, an inner side edge, and an underside. A neck support pad may be disposed between the left arm support pad and the right arm support pad, the neck support pad having a first end and a second end. A tummy support pad may be disposed below the left arm support pad and the right arm support pad. A plurality of adjustable straps may be included, wherein at least one adjustable strap may connect the neck support pad to at least one of the arm support pads, and at least one adjustable strap may connect the tummy support pad to at least one of the arm support pads. The adjustable straps may be releasably connected and adjusted to accommodate different body dimensions.

A method of using a corrective device to relieve neck, shoulder, and upper back pain may comprise placing a corrective device on a flat surface, the corrective device comprising a left arm support pad, a right arm support pad, a neck support pad disposed between the arm support pads, a tummy support pad disposed below the arm support pads, and a plurality of adjustable straps. The method may include positioning a user face down between the left arm support pad and the right arm support pad. The method may include placing the user's left arm on the left arm support pad and the user's right arm on the right arm support pad with the user's elbows positioned on the respective arm support pads. The method may include positioning the neck support pad under the user's chin and above the user's throat. The method may include positioning the tummy support pad under the user's navel. The method may include adjusting the adjustable straps to accommodate the user's body dimensions.

A corrective device may comprise a pair of arm support pads configured to support a user's arms. A neck support pad may be configured to support a user's chin. A tummy support pad may be configured to support a user's abdomen. A plurality of adjustable straps may connect the support pads. The corrective device may be configured to be used while the user is lying face down with arms positioned on the arm support pads, chin positioned on the neck support pad, and abdomen positioned on the tummy support pad.

A corrective device for relieving pain in the neck, shoulder, and upper back regions may comprise a left arm support pad having an upper end, a lower end, a first angled edge, a second angled edge, an inner side edge, and an underside. A right arm support pad may have an upper end, a lower end, a first angled edge, a second angled edge, an inner side edge, and an underside. A neck support pad may be positioned between the upper ends of the left and right arm support pads. A tummy support pad may be positioned between the lower ends of the left and right arm support pads. A first adjustable strap may be attached to a first end of the neck support pad. A second adjustable strap may be attached to a second end of the neck support pad. A third adjustable strap may be attached to a first end of the tummy support pad and extending to the underside of the left arm support pad. A fourth adjustable strap may be attached to a second end of the tummy support pad and extending to the underside of the right arm support pad.

### BRIEF DESCRIPTION OF DRAWINGS

The present disclosure may be better understood, and its numerous features and advantages made apparent to those skilled in the art by referencing the accompanying drawings. The use of the same reference symbols in different drawings indicates similar or identical items. The accompanying drawings, which are incorporated in and constitute a part of this specification, illustrate embodiments of the disclosure and, together with a general description of the disclosure given above, and the detailed description given below, serve to explain the principles of the disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. The present disclosure, in accordance with one or more various embodiments, is described in detail with reference to the following figures. The drawings are provided for purposes of illustration only and merely depict typical or example embodiments of the disclosure. These drawings are provided to facilitate the reader's understanding of the disclosure and shall not be considered limiting of the breadth, scope, or applicability of the disclosure.

The accompanying drawings, which are incorporated in and constitute a part of this specification, illustrate one or more embodiments of the invention and, together with the description, explain the invention. The present disclosure will become more fully understood from the detailed description and the accompanying drawings, wherein:

FIG. 1 is a top perspective view of the corrective device of the present subject matter.

FIG. 2 is a schematic view of the corrective device of the present subject matter.

FIG. 3 is a top perspective view of the corrective device of the present subject matter in operation.

FIG. 4 is a top perspective view of the corrective device of the present subject matter showing a preferred embodiment of the neck support pad.

### DESCRIPTION OF THE INVENTION

In the following description, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements and are not drawn to scale.

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well-known structures and components are shown in block diagram form in order to avoid obscuring such concepts.

The invention will be described in detail with reference to certain preferred embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention. The features and advantages of the present invention may be better understood with reference to the drawings and discussions that follow.

Throughout this specification and the claims, the terms “comprise,” “comprising,” “include,” “including,” and the like are to be understood to imply the inclusion of stated elements but not the exclusion of any other elements. The term “exemplary” is used in the sense of “example” rather than “ideal” or “model.” As used herein, the terms “about” or “approximately” apply to all numeric values, whether or not explicitly indicated. These terms generally refer to a range of numbers that one of skill in the art would consider equivalent to the recited values (i.e., having the same function or result).

Before the present articles, systems, apparatuses, and/or methods are disclosed and described, it is to be understood that they are not limited to specific methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

### Definitions

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise. As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

It is understood that the apparatuses and systems disclosed herein have certain functions. Disclosed herein are certain structural requirements for performing the disclosed functions, and it is understood that there are a variety of structures that can perform the same function that are related to the disclosed structures, and that these structures will typically achieve the same result. The following description of various embodiments is merely exemplary in nature and is in no way intended to limit the disclosure, its application, or uses.

The following description is merely illustrative in nature and is in no way intended to limit the disclosure, its application, or uses. For purposes of clarity, the same reference numbers will be used in the drawings to identify similar elements. As used herein, the phrase “at least one of” A, B, and C should be construed to mean a logical (A or B or C), using a non-exclusive logical OR. It should be understood that steps within a method may be executed in different order without altering the principles of the present disclosure.

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

In the following description, numerous specific details are set forth to clearly describe various specific embodiments disclosed herein. One skilled in the art, however, will understand that the presently claimed invention may be practiced without all of the specific details discussed below. In other instances, well known features have not been described so as not to obscure the invention.

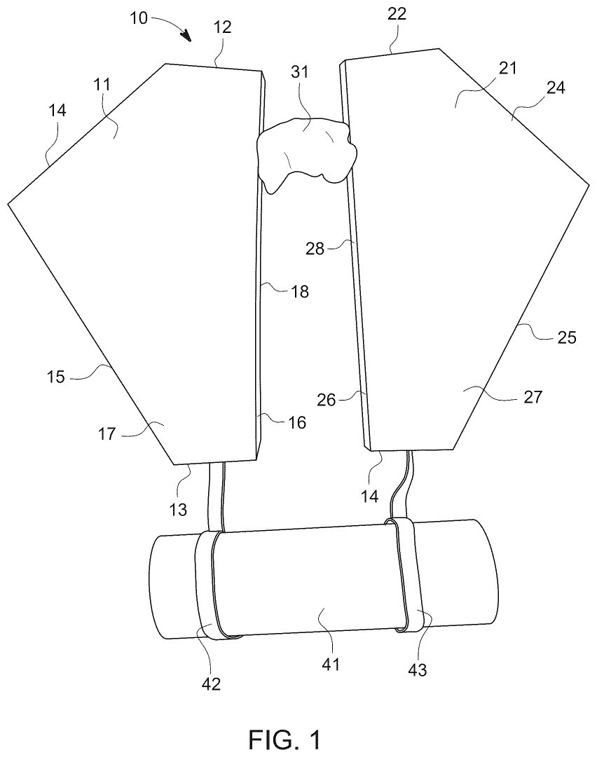

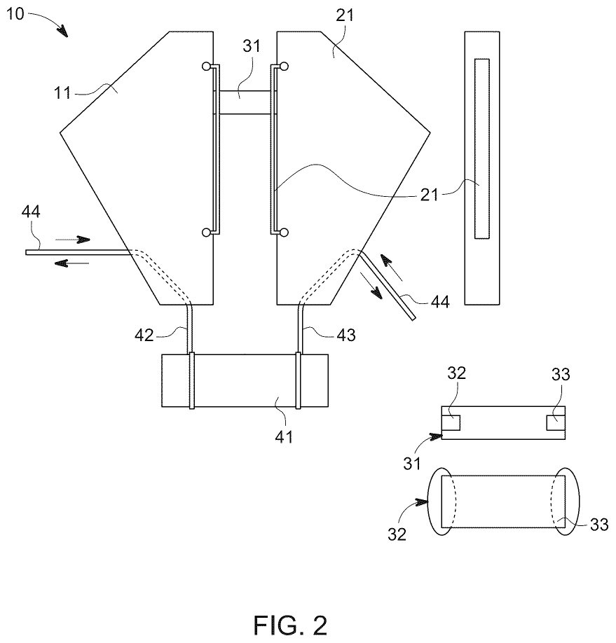

The corrective device of the present subject matter comprises four foam support pads and four adjustable strap members. Referring now to FIG. 1 and FIG. 2, the corrective device 10 comprises a left arm support pad 11, a right arm support pad 21, a neck support pad 31 and a tummy support pad 41. Each of the left and right arm support pads 11 and 21 may be about twenty-four inches (24″) in length and about eighteen inches (18″) at the widest point. Left arm support pad 11 may have an upper end 12 which may be about six inches (6″) wide, a lower end 13 which may be about six inches (6″) wide, a first angled edge 14 which may extend outwardly and downwardly to a point about nine inches (9″) from the upper end 12, a second angled edge 15 which may extend inwardly and downwardly to the lower end 13, an inner side edge 16, and an under side 17. Similarly, right support pad 21 may have an upper end 22 which may be about six inches (6″) wide, a lower end 23 which may be about six inches (6″) wide, a first angled edge 24 which may extend outwardly and downwardly to a point about nine inches (9″) from the upper end 22, a second angled edge 25 which may extend inwardly and downwardly to the lower end 23, an inner side edge 26, and an underside 27. An adjustable strap 18 may be attached to the inner side edge 16 of the left arm support pad 11 and an adjustable strap 28 may be attached to the inner side edge 26 of the right arm support pad 21.

The neck support pad 31 may be in the form of a foam element disposed between the left arm support pad 11 and the right support pad 21 and may comprise adjustable straps 32 and 33 attached to either end of the neck support pad. The neck support pad 31 may be a foam pad measuring about eight inches (8″) in length, about three inches (3″) in width and about one-and-one-half inches (1.5″) in depth.

The tummy support pad 41 may be in the form of a solid foam roll having a diameter of about five inches (5″) and may be about sixteen inches (16″) in length. Attached near each end of the tummy support pad 41 may be adjustable straps 42 and 43. Adjustable strap 42 may extend upwardly to the under side 17 of the left arm support pad 11 to a point about six inches (6″) from the lower end 13 and then may extend outwardly from the second angled edge 15. Similarly, adjustable strap 43 may extend upwardly to the underside 27 of the right arm support pad 21 to a point about six inches (6″) from the lower end 23 and then may extend outwardly from the second angled edge 25. In one embodiment, each of the adjustable straps 42 and 43 may have complementing connecting means 44 at each of the distal ends such that the adjustable straps can be removably connected and adjusted.

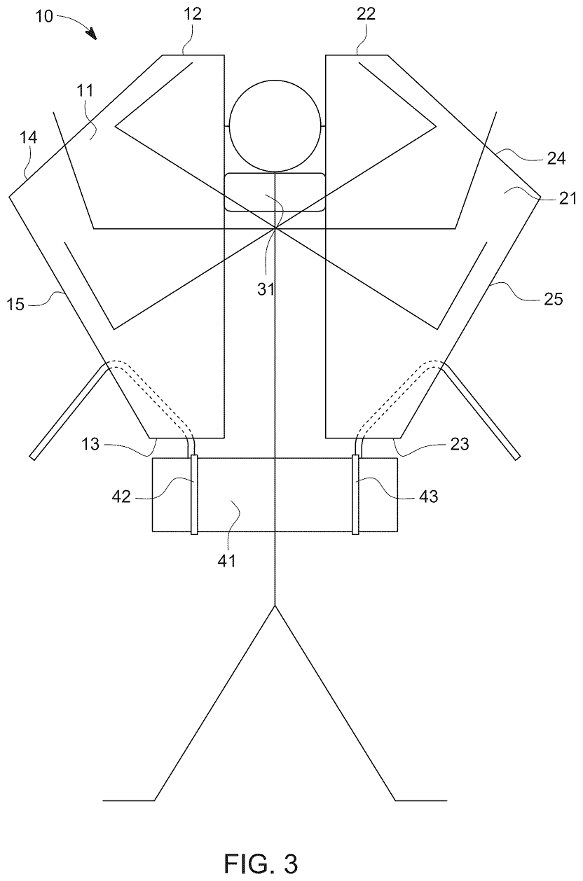

Referring now to FIG. 3, in operation, the correcting device of the present subject matter may be placed on a flat surface and the user may lie face down between the left arm support pad 11 and the right arm support pad 21, and may position the user's left arm on the left arm support pad and the user's right arm on the right arm support pad with the user's elbows being positioned on the respective left arm and right arm support pads. The neck support pad 31 may be positioned under the user's chin, above the user's throat and the tummy support pad 41 may be positioned under the user's navel. The adjustable straps provided on the neck support pad and the tummy support pad may be releasably connected and adjusted to the user's body measurements. The arm support pads can be adjusted for different arm positions as shown in FIG. 3, with the user's elbows always remaining on the arm support pads.

The corrective device may provide relief from neck pain, shoulder pain and upper back pain. It may correct certain physical ailments such as Kyphosis Hump (e.g. Widow's Hump or Dowager's Hump) and Rounded Shoulders.

The corrective device may be constructed from foam material or other suitable materials that may provide adequate support and comfort to the user. In various embodiments, the foam used for the arm support pads 11 and 21, neck support pad 31, and tummy support pad 41 may be high-density foam, memory foam, or other types of foam that may provide sufficient firmness while still conforming to the user's body. The density and firmness of the foam used for each support pad may vary depending on the specific function of each pad.

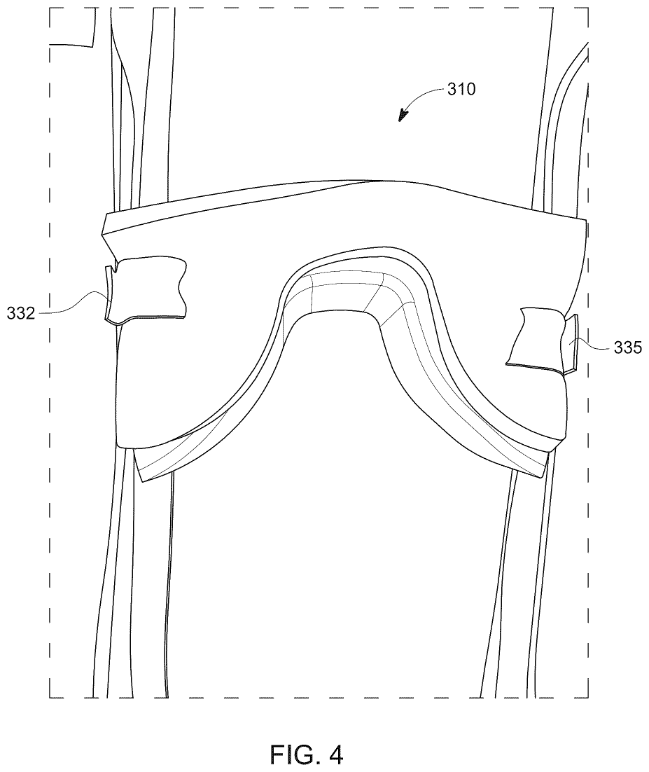

Referring to FIG. 4, in a preferred embodiment, the neck support pad 31 may be contoured to better accommodate the user's chin and neck area. The neck support pad 31 may have a curved upper surface 34 that may conform to the shape of the user's chin. The curved upper surface 34 may help distribute pressure evenly across the chin area and may prevent discomfort during use. The neck support pad 31 may also have tapered edges 35 that may provide a gradual transition from the support pad to the surrounding area, which may enhance comfort during use.

The adjustable straps 18, 28, 32, 33, 42, and 43 may be made from nylon, elastic material, or other suitable materials that may provide adequate strength and flexibility. Each adjustable strap may include complementary connecting means 44 such as hook and loop fasteners, buckles, snaps, or other suitable fastening mechanisms that may allow for secure connection and easy adjustment. The connecting means 44 may be designed to withstand repeated use without significant degradation in performance.

In some embodiments, the undersides 17 and 27 of the left arm support pad 11 and right arm support pad 21, respectively, may include non-slip surfaces or materials that may help prevent the corrective device 10 from sliding on smooth floors or surfaces during use. The non-slip surfaces may be in the form of textured patterns, rubber strips, or other suitable materials that may increase friction between the corrective device and the supporting surface.

The corrective device 10 may be configured to accommodate users of different sizes and body types. The adjustable straps 18, 28, 32, 33, 42, and 43 may be of sufficient length to accommodate larger users, and the connecting means 44 may allow for significant adjustment to fit smaller users as well. In some embodiments, the arm support pads 11 and 21 may be available in different sizes to better accommodate users with different arm lengths or shoulder widths.

For enhanced portability, the corrective device 10 may be designed to be foldable or collapsible. The arm support pads 11 and 21 may be hinged or otherwise connected to the neck support pad 31 in a manner that may allow the device to be folded into a more compact configuration for storage or transport. The tummy support pad 41 may be detachable from the arm support pads via the adjustable straps 42 and 43 to further facilitate compact storage.

In operation, the user may place the corrective device 10 on a flat surface such as a floor, bed, or exercise mat. The user may then lie face down between the left arm support pad 11 and the right arm support pad 21. The user's left arm may be positioned on the left arm support pad 11 and the user's right arm may be positioned on the right arm support pad 21, with the user's elbows positioned on the respective arm support pads. The position of the elbows on the arm support pads may be a key factor in the effectiveness of the corrective device.

The neck support pad 31 may be positioned under the user's chin and above the user's throat. The position of the neck support pad 31 may be adjusted using the adjustable straps 32 and 33 to ensure proper support and comfort for the user's neck. The tummy support pad 41 may be positioned under the user's navel, and the adjustable straps 42 and 43 may be used to secure the tummy support pad in the proper position relative to the arm support pads.

The user may adjust the position of their arms on the arm support pads 11 and 21 to achieve different therapeutic effects. As shown in FIG. 3, the arm support pads may accommodate various arm positions while maintaining the user's elbows on the support pads. The ability to adjust arm positions may allow the user to target specific areas of discomfort or to gradually increase the intensity of the stretch as their condition improves.

The corrective device 10 may be used for varying durations depending on the user's needs and comfort level. Initially, users may start with shorter sessions of 5-10 minutes and gradually increase the duration as they become more comfortable with the device. Regular use of the corrective device may help correct postural issues such as Kyphosis Hump (also known as Widow's Hump or Dowager's Hump) and Rounded Shoulders by gently stretching and realigning the muscles of the neck, shoulders, and upper back.

The corrective device 10 may also be used in conjunction with other therapeutic modalities such as physical therapy exercises, massage, or heat/cold therapy to enhance its effectiveness. Healthcare providers such as physical therapists, chiropractors, or pain management specialists may incorporate the corrective device into treatment plans for patients with neck, shoulder, or upper back pain.

In some embodiments, the corrective device 10 may include additional features such as heating or cooling elements embedded within the support pads to provide thermal therapy in conjunction with the postural correction. The heating or cooling elements may be removable or may be integrated into the structure of the support pads.

The corrective device 10 may be covered with removable, washable covers to maintain hygiene, especially in settings where the device may be used by multiple individuals. The covers may be made from soft, breathable fabrics that may enhance comfort during use while protecting the foam pads from moisture, oils, and other contaminants.

For users who may experience discomfort in the chest area while lying prone, the corrective device 10 may optionally include additional chest support pads that may be positioned between the arm support pads 11 and 21. The chest support pads may help distribute pressure more evenly across the chest area and may reduce discomfort for users with larger chest sizes or those with chest-related conditions.

The corrective device 10 may be used in various settings including home, physical therapy clinics, chiropractic offices, fitness centers, or other locations where therapeutic devices may be utilized. The simplicity of the design and operation may allow users to effectively use the device without extensive training or supervision, although initial guidance from a healthcare provider may be beneficial for optimal results.

The corrective device 10 may provide several technical advantages over conventional therapeutic devices for neck, shoulder, and upper back pain. The specific design and arrangement of the support pads may work together to create a biomechanically advantageous position for the user's spine, shoulders, and neck. When the user lies face down with their arms positioned on the arm support pads 11 and 21, with elbows properly positioned on these pads, a gentle stretching force may be applied to the muscles of the upper back, shoulders, and neck. This stretching force may help to counteract the forward-hunched posture that may be associated with conditions such as Kyphosis Hump and Rounded Shoulders.

The trapezoidal shape of the arm support pads 11 and 21, with their first angled edges 14 and 24 extending outwardly and downwardly from the upper ends 12 and 22, and their second angled edges 15 and 25 extending inwardly and downwardly to the lower ends 13 and 23, may be particularly advantageous. This shape may allow for optimal positioning of the user's arms in a manner that may effectively target the muscles responsible for maintaining proper posture. The width of approximately eighteen inches at the widest point may provide ample support for users with varying shoulder widths.

The positioning of the neck support pad 31 between the upper ends of the arm support pads may provide crucial support for the user's cervical spine. By supporting the chin above the throat, the neck support pad 31 may help maintain the cervical spine in a neutral position, which may reduce strain on the neck muscles and may help alleviate neck pain. The dimensions of the neck support pad-approximately eight inches in length, three inches in width, and one-and-one-half inches in depth—may be optimized to provide adequate support without causing discomfort.

The curved upper surface 34 of the neck support pad 31, as shown in FIG. 4, may represent a significant improvement over flat support surfaces. This contoured design may distribute pressure more evenly across the chin area, which may enhance comfort during use and may allow for longer therapeutic sessions. The tapered edges 35 may further contribute to user comfort by providing a gradual transition from the support pad to the surrounding area.

The tummy support pad 41, positioned below the arm support pads, may serve multiple functions. Its cylindrical shape with a diameter of approximately five inches may elevate the user's abdomen slightly, which may help maintain proper spinal alignment during use. This elevation may reduce pressure on the lumbar spine and may allow for a more comfortable prone position. The position of the tummy support pad under the user's navel may be particularly effective for achieving the desired therapeutic effects.

The system of adjustable straps 18, 28, 32, 33, 42, and 43 may provide significant advantages in terms of customization and fit. The ability to adjust these straps to accommodate different body dimensions may allow the corrective device 10 to be effective for a wide range of users. The releasable connections provided by the complementary connecting means 44 may facilitate easy adjustment and may allow users to fine-tune the device to their specific needs.

The use of high-density foam or memory foam for the support pads may provide an optimal balance of support and comfort. The foam material may conform to the user's body while still maintaining sufficient firmness to provide the necessary therapeutic effects. This combination of support and comfort may encourage regular use of the device, which may be essential for achieving long-term benefits.

The non-slip surfaces that may be included on the undersides 17 and 27 of the arm support pads may enhance safety and effectiveness by preventing unwanted movement of the device during use. This stability may allow users to focus on achieving the proper therapeutic position without concerns about the device shifting or sliding.

The ability to adjust arm positions while maintaining the elbows on the arm support pads, as shown in FIG. 3, may provide users with the flexibility to target specific areas of discomfort or to gradually increase the intensity of the stretch as their condition improves. This adaptability may make the corrective device 10 suitable for users at different stages of recovery or with varying levels of flexibility.

The portability features, such as the potential for the device to be foldable or collapsible, may enhance its practicality for home use. The ability to store the device compactly when not in use may encourage regular utilization, which may be essential for achieving and maintaining therapeutic benefits.

The corrective device 10 may be particularly effective for addressing specific postural issues such as Kyphosis Hump and Rounded Shoulders. By gently stretching and realigning the muscles of the neck, shoulders, and upper back, the device may help counteract the forward-hunched posture that may characterize these conditions. Regular use of the device may lead to improved posture and reduced pain in the affected areas.

The simplicity of the design and operation may allow users to effectively use the device without extensive training or supervision. This accessibility may make the corrective device 10 suitable for home use as part of a self-care regimen for neck, shoulder, and upper back pain. The device may also be incorporated into professional therapeutic settings as a complement to other treatment modalities.

The corrective device 10 may also be designed to be adjustable to accommodate different body sizes and shapes. In some embodiments, the arm support pads 11 and 21 may be made from layered foam materials with different densities to provide optimal support and comfort. The top layer may be made from softer memory foam that may conform to the user's arms, while the bottom layer may be made from firmer high-density foam that may provide structural support and durability.

The neck support pad 31 may be designed with additional features to enhance its therapeutic effectiveness. In some embodiments, the neck support pad 31 may include a central depression or groove that may accommodate the user's chin more comfortably. This feature may help maintain proper alignment of the cervical spine during use. The depth of this depression may be approximately one-half inch (0.5″) to provide adequate support without causing discomfort.

In alternative embodiments, the neck support pad 31 may be height-adjustable to accommodate users with different neck lengths or to provide varying levels of cervical extension. This height adjustment may be achieved through the use of removable foam layers or through an adjustable mechanism integrated into the structure of the neck support pad. The ability to adjust the height of the neck support pad may allow users to gradually increase the degree of cervical extension as their condition improves.

The tummy support pad 41 may be designed with a slight concave curvature on its upper surface to better accommodate the contours of the user's abdomen. This curvature may help distribute pressure more evenly across the abdominal area and may reduce discomfort during extended use. The concave curvature may have a depth of approximately one-half inch (0.5″) at the center of the tummy support pad.

In some embodiments, the tummy support pad 41 may be divided into sections that may be adjusted independently to provide customized support for different body types. These sections may be connected by flexible joints or hinges that may allow the tummy support pad to conform more closely to the user's body. This feature may be particularly beneficial for users with non-typical body shapes or those with specific abdominal conditions that may require specialized support.

The adjustable straps 18, 28, 32, 33, 42, and 43 may be designed with ergonomic features to enhance comfort and ease of use. The straps may have padded sections where they may come into contact with the user's body to prevent pressure points or irritation.

The connecting means 44 may be designed to be easily manipulated even by users with limited hand dexterity or strength, which may be important for elderly users or those with arthritic conditions.

In some embodiments, the adjustable straps may include measurement markings or indicators that may help users maintain consistent settings across multiple sessions. These markings may also facilitate communication between users and healthcare providers regarding the optimal settings for specific therapeutic goals. The ability to precisely replicate strap settings may enhance the consistency and effectiveness of the therapeutic benefits.

The arm support pads 11 and 21 may be designed with additional features to enhance their therapeutic effectiveness. In some embodiments, the arm support pads may include raised edges or contoured surfaces that may help guide the user's arms into optimal positions. These features may be particularly helpful for users who may be new to the device or who may have difficulty maintaining proper arm positions during use.

The corrective device 10 may be designed with features that may enhance its usability in various environments. The materials used for the support pads and straps may be selected for their resistance to moisture, heat, and cold, which may allow the device to be used in conjunction with heat or cold therapy. The materials may also be selected for their ease of cleaning and maintenance, which may be important for devices used in clinical settings or by multiple users.

In one or more embodiments, a corrective device may comprise a left arm support pad and a right arm support pad. Each arm support pad may have an upper end, a lower end, a first angled edge extending outwardly and downwardly from the upper end, a second angled edge extending inwardly and downwardly to the lower end, an inner side edge, and an underside. The dimensions of each arm support pad may be approximately twenty-four inches in length and approximately eighteen inches at its widest point. A neck support pad may be disposed between the left arm support pad and the right arm support pad. The neck support pad may have a first end and a second end. The neck support pad may be a foam pad measuring approximately eight inches in length, approximately three inches in width, and approximately one-and-one-half inches in depth. A tummy support pad may be disposed below the left arm support pad and the right arm support pad. The tummy support pad may be a solid foam roll having a diameter of approximately five inches and a length of approximately sixteen inches. A plurality of adjustable straps may connect the various support pads. At least one adjustable strap may connect the neck support pad to at least one of the arm support pads, and at least one adjustable strap may connect the tummy support pad to at least one of the arm support pads. The adjustable straps may be releasably connected and adjusted to accommodate different body dimensions. The adjustable straps may include complementary connecting means at their distal ends to allow for removable connection and adjustment. A first adjustable strap may be attached to a first end of the neck support pad, and a second adjustable strap may be attached to a second end of the neck support pad. A third adjustable strap may be attached to a first end of the tummy support pad and may extend to the underside of the left arm support pad. The third adjustable strap may extend from the underside of the left arm support pad to a point approximately six inches from the lower end of the left arm support pad and then may extend outwardly from the second angled edge of the left arm support pad. A fourth adjustable strap may be attached to a second end of the tummy support pad and may extend to the underside of the right arm support pad. The fourth adjustable strap may extend from the underside of the right arm support pad to a point approximately six inches from the lower end of the right arm support pad and then may extend outwardly from the second angled edge of the right arm support pad.

In some embodiments, the corrective device 10 may include visual guides or markers that may help users achieve proper positioning. These guides may include color-coded areas, alignment marks, or instructional graphics printed directly on the support pads. These visual cues may help users maintain consistent positioning across multiple sessions and may reduce the need for supervision or assistance during use.

The corrective device 10 may be designed to work in conjunction with other therapeutic devices or modalities. In some embodiments, the support pads may include pockets or attachment points for hot/cold packs, TENS unit electrodes, or other therapeutic accessories. This integration may enhance the overall therapeutic effect by combining postural correction with other treatment modalities.

The biomechanical principles underlying the design of the corrective device 10 may be based on the concept of gentle, sustained stretching of the muscles and connective tissues that may contribute to poor posture. When the user lies face down with their arms positioned on the arm support pads 11 and 21 and their chin on the neck support pad 31, the muscles of the upper back, shoulders, and neck may be placed in a stretched position. This stretched position may be maintained for an extended period, which may help lengthen shortened muscles and may improve flexibility in the affected areas.

The specific dimensions and arrangement of the support pads may be designed to create optimal biomechanical leverage for correcting postural issues. The width of the arm support pads at their widest point (approximately eighteen inches) may provide sufficient leverage to effectively stretch the muscles of the upper back and shoulders. The distance between the arm support pads and the neck support pad may be optimized to create an effective stretch without causing discomfort or strain.

The trapezoidal shape of the arm support pads 11 and 21 may provide biomechanical advantages beyond simple support. The angled edges may allow for a range of arm positions that may target different muscle groups while maintaining the elbows on the support pads. This versatility may allow users to customize their therapeutic experience based on their specific needs and comfort levels.

The cylindrical shape of the tummy support pad 41 may serve to elevate the user's abdomen slightly, which may help maintain proper spinal alignment during use. This elevation may reduce pressure on the lumbar spine and may allow for a more effective stretch of the upper back and shoulder muscles. The diameter of the tummy support pad (approximately five inches) may be optimized to provide sufficient elevation without causing discomfort or strain on the lower back.

The adjustable straps 18, 28, 32, 33, 42, and 43 may play a crucial role in the biomechanical effectiveness of the corrective device 10. By securing the support pads in optimal positions relative to the user's body, the straps may help maintain the therapeutic stretching force throughout the duration of use. The ability to adjust these straps may allow users to fine-tune the intensity of the stretch based on their comfort level and therapeutic goals.

The materials used for the support pads may be selected for their ability to provide firm support while still conforming to the user's body. This combination of support and comfort may be essential for maintaining the therapeutic position for extended periods. The density and resilience of the foam materials may be optimized to provide the necessary support without causing pressure points or discomfort.

In some embodiments, the corrective device 10 may include additional features to enhance user comfort and compliance. These features may include aromatherapy elements, such as lavender-scented covers, that may promote relaxation during use. The device may also include audio guidance components that may lead users through breathing exercises or relaxation techniques while using the device. These comfort-enhancing features may encourage regular use of the device, which may be essential for achieving long-term therapeutic benefits.

The corrective device 10 may be particularly effective for addressing specific postural issues such as Kyphosis Hump and Rounded Shoulders. These conditions may be characterized by forward-hunched posture, which may result from prolonged sitting, poor ergonomics, or age-related changes in the spine. By gently stretching and realigning the muscles of the neck, shoulders, and upper back, the corrective device may help counteract these postural deviations and may promote better alignment of the spine.

Regular use of the corrective device 10 may lead to improved posture and reduced pain in the affected areas. The specific therapeutic benefits may include increased flexibility in the muscles of the upper back and shoulders, improved alignment of the cervical and thoracic spine, and reduced tension in the neck muscles. These improvements may translate to better overall posture, reduced pain, and enhanced quality of life for users.

The corrective device 10 may be used as part of a comprehensive approach to posture correction and pain management. While the device may provide significant benefits on its own, its effectiveness may be enhanced when used in conjunction with other therapeutic modalities such as physical therapy exercises, massage, or ergonomic improvements in the user's daily environment. Healthcare providers may incorporate the corrective device into treatment plans for patients with neck, shoulder, or upper back pain as part of a multi-faceted approach to care.

The simplicity of the design and operation of the corrective device 10 may make it accessible to a wide range of users, including those who may not have access to regular professional care. The device may be used at home as part of a self-care regimen, which may empower users to take an active role in managing their posture and pain. This accessibility may be particularly valuable for users in underserved areas or those with limited mobility who may have difficulty accessing traditional healthcare services.

In conclusion, the corrective device 10 may represent a significant advancement in the field of non-invasive therapies for neck, shoulder, and upper back pain. Its unique design, which may incorporate biomechanical principles of gentle, sustained stretching, may provide effective relief for conditions such as Kyphosis Hump and Rounded Shoulders. The device's adjustability, portability, and ease of use may make it a practical option for a wide range of users, from those seeking relief from occasional discomfort to those managing chronic postural issues. With regular use, the corrective device may help users achieve improved posture, reduced pain, and enhanced quality of life.

While several illustrative embodiments of the invention have been shown and described, numerous variations and alternative embodiments will occur to those skilled in the art. Such variations and alternative embodiments are contemplated and can be made without departing from the scope of the invention as defined in the appended claims.

What is claimed is:

1. A method of using a corrective device to relieve neck, shoulder, and upper back pain, the method comprising:
placing a corrective device on a flat surface, the corrective device comprising a left arm support pad, a right arm support pad, a neck support pad disposed between the arm support pads, a tummy support pad disposed below the arm support pads, and a plurality of adjustable straps;
positioning a user face down between the left arm support pad and the right arm support pad;
placing the user's left arm on the left arm support pad and the user's right arm on the right arm support pad with user's elbows positioned on the respective arm support pads;
positioning the neck support pad under user's chin and above the user's throat;
positioning the tummy support pad under user's navel; and
adjusting the adjustable straps to accommodate user's body dimensions.

2. The method of claim 1, wherein the arm support pads can be adjusted for different arm positions while maintaining the user's elbows on the arm support pads.

3. The method of claim 1, wherein the corrective device provides relief from at least one of neck pain, shoulder pain, and upper back pain.

4. The method of claim 1, wherein the corrective device is configured to correct kyphosis hump.

5. The method of claim 1, wherein the corrective device is configured to correct rounded shoulders.

6. The method of claim 1, wherein the adjustable straps comprise a first adjustable strap attached to a first end of the neck support pad, a second adjustable strap attached to a second end of the neck support pad, a third adjustable strap attached to a first end of the tummy support pad, and a fourth adjustable strap attached to a second end of the tummy support pad.

7. A corrective device comprising:
a pair of arm support pads configured to support a user's arms;
a neck support pad configured to support a user's chin;
a tummy support pad configured to support a user's abdomen; and
a plurality of adjustable straps connecting the support pads,
wherein the corrective device is configured to be used while the user is lying face down with arms positioned on the arm support pads, chin positioned on the neck support pad, and abdomen positioned on the tummy support pad.

8. The corrective device of claim 7, wherein each arm support pad has a trapezoidal shape with an upper end, a lower end, and angled side edges.

9. The corrective device of claim 7, wherein the neck support pad is positioned between upper ends of the arm support pads.

10. The corrective device of claim 7, wherein the tummy support pad is positioned between lower ends of the arm support pads.

11. The corrective device of claim 7, wherein the adjustable straps include complementary connecting means at their distal ends to allow for removable connection and adjustment.

12. The corrective device of claim 7, wherein the arm support pads, neck support pad, and tummy support pad are made of foam.

13. The corrective device of claim 7, wherein the tummy support pad is cylindrical in shape.

14. The corrective device of claim 7, wherein the corrective device is configured to correct at least one of Kyphosis Hump and Rounded Shoulders.

15. A corrective device for relieving pain in neck, shoulder, and upper back regions, the device comprising:
a left arm support pad having an upper end, a lower end, a first angled edge, a second angled edge, an inner side edge, and an underside;
a right arm support pad having an upper end, a lower end, a first angled edge, a second angled edge, an inner side edge, and an underside;
a neck support pad positioned between the upper ends of the left and right arm support pads;
a tummy support pad positioned between the lower ends of the left and right arm support pads;
a first adjustable strap attached to a first end of the neck support pad;
a second adjustable strap attached to a second end of the neck support pad;
a third adjustable strap attached to a first end of the tummy support pad and extending to the underside of the left arm support pad; and
a fourth adjustable strap attached to a second end of the tummy support pad and extending to the underside of the right arm support pad.

16. The corrective device of claim 15, wherein the third adjustable strap extends from the underside of the left arm support pad to a point approximately six inches from the lower end of the left arm support pad and then extends outwardly from the second angled edge of the left arm support pad.

17. The corrective device of claim 15, wherein the fourth adjustable strap extends from the underside of the right arm support pad to a point approximately six inches from the lower end of the right arm support pad and then extends outwardly from the second angled edge of the right arm support pad.

18. The corrective device of claim 15, wherein the tummy support pad is a solid foam roll having a diameter of approximately five inches and a length of approximately sixteen inches.

19. The corrective device of claim 15, wherein the adjustable straps can be releasably connected and adjusted to accommodate different body dimensions.

20. The corrective device of claim 15, wherein the arm support pads can be adjusted for different arm positions while maintaining a user's elbows on the arm support pads.
